(19) United States
(12) Patent Application Publication
(10) Pub. No.: US 2009/0093738 A1
(43) Pub. Date: Apr. 9, 2009

(54) DEVICE AND METHOD FOR MONITORING A TREATMENT AREA

(75) Inventors: Adnan I. Merchant, Fremont, CA (US); Joel C. Colburn, Walnut Creek, CA (US)

Correspondence Address: FULWIDER PATTON - CABOCHON AESTHETICS, 6060 CENTER DRIVE, TENTH FLOOR, LOS ANGELES, CA, 90045, US

(73) Assignee: CABOCHON AESTHETICS, INC., Menlo Park, CA (US)

(21) Appl. No.: 12/247,879

(22) Filed: Oct. 8, 2008

Related U.S. Application Data
(60) Provisional application No. 60/978,646, filed on Oct. 9, 2007.

(51) Int. Cl. A61N 7/02; A61N 7/00; A61K 49/00

(52) U.S. Cl. 601/3; 601/2; 424/9.1

(57) ABSTRACT

A method for monitoring the amount of treatment delivered by a treatment device, comprising: providing a treatment device including at least one activation element; applying a reactive agent to a treatment area, the reactive agent reacting to energy delivered by the at least one activation element; actuating the treatment device and the activation element to deliver treatment.

### CROSS-REFERENCES TO RELATED APPLICATIONS

This application claims priority from U.S. Provisional Application No. 60/978,646, filed Oct. 9, 2007 incorporated by reference in its entirety.

### FIELD OF THE INVENTION

The invention relates to devices and compositions useful for monitoring or tracking whether a particular region has been treated and/or the amount of treatment that a given area has received. More particularly, the invention relates to monitoring devices and compositions which are useful in combination with ultrasound.

### BACKGROUND

Current methods for monitoring whether a given area has previously been treated are cumbersome, complex, expensive to implement, or are not amenable for use in conjunction with therapeutic and diagnostic ultrasound. Accordingly, what is needed is a method and apparatus for monitoring treatment of a treatment area which is reliable, inexpensive and easy to implement.

### SUMMARY OF THE INVENTION

Disclosed is a method for monitoring the amount of treatment delivered by a treatment device. The method includes providing a treatment device including at least one activation element and at least one reactive agent which is activatable by the activation element. The reactive agent is applied to a treatment area, and actuating the treatment device and the activation element to deliver treatment.

The reactive agent may provide indication of whether a given treatment area has been treated. The reactive agent may provide indication of the amount of treatment provided to a given treatment area. The reactive agent may provide indication of the peak temperature of the treatment area. The reactive agent may provide indication of a duration of treatment within the treatment zone.

In the aforementioned embodiment, the treatment device may be the activation element, or the activation element may be a separate device.

According to one embodiment, the reactive agent is one of a thermochrome and a chromaphore.

Also disclosed is a system for monitoring treatment parameters, including a treatment device for delivering treatment to a treatment area, at least one activation element operably coupled with the treatment device such that the activation element is actuated each time the treatment device is actuated, and a reactive agent applied to the treatment area and responsive to energy transmitted from the activation element. The reactive agent may be a thermochrome or a chromaphore. The reactive agent is responsive to at least one of heat and light.

The reactive agent provides indication of whether a given treatment area has been treated, and/or the amount of treatment provided to a given treatment area, and/or the peak temperature of the treatment area. The reactive agent provides indication of a duration of treatment within the treatment zone.

Also disclosed is a composition, including a reactive agent in a nonreactive acoustically transparent carrier wherein the reactive agent is responsive to energy exposure and provides indication of at least one of a quantity and intensity of energy exposure.

### BRIEF DESCRIPTION OF THE DRAWINGS

FIG. 1 is a block diagram of a treatment device according to the present invention.

### DETAILED DESCRIPTION OF THE PREFERRED EMBODIMENTS

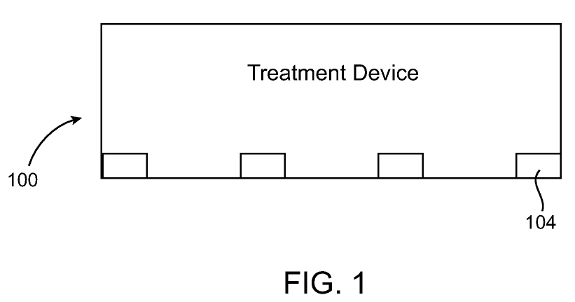

FIG. 1 depicts a treatment or energy delivery device 100. According to some embodiments treatment device 100 is an ultrasound transducer; however, treatment device 100 is not limited to ultrasound and may provide any desired treatment, e.g., radio-frequency (RF), laser or the like.

According to some embodiments, treatment device 100 includes one or more activation elements 104 operably coupled to device 100. Activation element 104 is an energy transmitting device (e.g. heat or laser/light) designed to work in cooperation with a reactive agent (not shown) applied to the treatment area such that a detectable characteristic of the reactive agent, e.g., color, changes in response to energy or the like transmitted from activation element 104.

Activation element 104 is preferably mounted and/or coupled to treatment device 100; however, if desired, activation element 104 may be a standalone device. Activation element 104 is coupled to treatment device 100 such that activation element 104 is actuated each time treatment device 100 is actuated. Alternatively, activation element 104 may be actuated for a duration corresponding to the duration of treatment by treatment device 100.

Optionally, treatment device 100 may be capable of varying the intensity of the treatment. Correspondingly, activation element 104 may be capable of modes of operation; i.e., varying the intensity of energy delivery, such that activation element 104 is responsive to the mode of operation of treatment device 100.

According to some embodiments a single reactive agent is used. But, according to other embodiments two or more reactive agents are provided. Each reactive agent may be a chromaphore, a thermochrome, or the like which reacts when exposed to light, heat, or other energy sources, e.g. RF, ultrasound or the like. Thus two or more thermochromes, two or more chromaphores, or a combination of thermochromes and chromaphores may be provided.

Thermochromic material is a temperature-sensitive compound that temporarily change color with exposure to heat. Typically, the thermochromic material contains thermochromic inks or dyes that come in two forms, liquid crystals and leuco dyes.

In operation, the reactive agent(s) are applied to the treatment area prior to treatment and change color or the like when exposed to heat and/or light from the either the treatment device, activation element(s) 104 or both the treatment device and activation elements. In the most basic form the reactive agent may have a binary (on/off) reaction indicative of whether an area has been treated. Alternatively, the reactive agent may provide indication of the duration of treatment or the intensity of the treatment, e.g., peak temperature, provided within the treatment area by changing hue or color.

The reactive agent may include two or more thermochromes or chromaphores, or a combination of thermochromes and chromaphores each of which react to a different level of treatment.

The reactive agent may for example provide indication of at least two performance criteria. More particularly the reactive agent may provide indication of peak temperature and/or duration of treatment.

Table 1 is a non-exhaustive list of chromaphores which may be used as part of the reactive agent. Table 2 is a non-exhaustive list of thermochromes which may be used as part of the reactive agent.

TABLE 1
Chromaphores
Anthocyanins
Chlorophyll
Hemoglobin
Hemocyanin
Phytochrome
Phycobilin
Bilirubin
β-carotene
Retinal
Azo compounds
Lycopene

TABLE 2
Thermochromes
Liquid Crystals
Cholesteryl nonanoate
Cyanobiphenyls.
Leuco Dyes
Spirolactones
Fluorans
Spiropyrans
Fulgides

The reactive agents may be included in or combined with a nonreactive carrier. For example, the reactive agent(s) may be provided in a cream, gel, or the like which is applied to the treatment area. Obviously, both the carrier and the reactive agent selected should be compatible with the energy or treatment delivered by treatment device 100. Thus, if treatment device 100 is an ultrasound transducer the carrier and the reactive agent should be compatible with ultrasound.

According to one embodiment the nonreactive gel is an acoustic coupling medium, such as ultrasound gel, castor oil or honey.

If the reactive agent includes a chromaphore then the treatment device or activation element 104 will include a light source such as a light emitting diode (LED) or the like. If treatment device 100 is a light source suitable for activating the reactive agent then a separate activation element 104 may be unnecessary and treatment device 100 will also serve as activation element 104. Alternatively, if treatment device 100 is a light source then activation element 104 could include a heat source.

If the reactive agent includes a thermochrome, then either treatment device 100 or activation element 104 will include a heat source such as a resistive heating element or the like. If treatment device 100 is a heat source suitable for activating the reactive agent then a separate activation element 104 may be unnecessary and treatment device 100 will also serve as activation element 104.

According to one embodiment, a method for monitoring the amount of treatment delivered by a treatment device is disclosed. The method includes providing a treatment device including at least one activation element. At least one reactive agent is applied to a treatment area, each reactive agent reacting to energy delivered by activation element. The treatment device and activation element are activated to deliver treatment, and the reactive element provides a visual indication responsive to energy from the activation element.

According to another embodiment, a system for monitoring treatment parameters is disclosed. The system includes a treatment device for delivering treatment to a treatment area, at least one activation element operably coupled with the treatment device such that the activation element is actuated each time the treatment device is actuated, and at least one reactive agent is applied to the treatment area, each said reactive agent responsive to energy transmitted from the activation element.

According to one embodiment, a method for monitoring the amount of treatment delivered by a treatment device is disclosed. The method includes providing a treatment device. At least one reactive agent is applied to a treatment area, each reactive agent reacting to energy delivered by the treatment device. The treatment device is activated to deliver treatment to the treatment area, and the reactive element provides a visual indication responsive to energy from the treatment device.

According to another embodiment, a system for monitoring treatment parameters is disclosed. The system includes a treatment device for delivering treatment to a treatment area, and at least one reactive agent is applied to the treatment area, each said reactive agent responsive to energy transmitted from the treatment device.

In any of the embodiments disclosed herein, the reactive agent may be one of a thermochrome and a chromaphore. The reactive agent may include more than one thermochrome or chromaphore. The reactive agent may include both a thermochrome and a chromaphore.

The reactive agent may provide indication of whether a given treatment area has been treated. The reactive agent may provide indication of the amount (duration) of treatment provided to a given treatment area. The reactive agent provides indication of the peak temperature of the treatment area. The reactive agent may provide indication of both the peak temperature within the treatment area and the duration of treatment provided to the treatment area.

According to one embodiment, where the treatment device is an ultrasound transducer and the reactive agent is nonreactive to ultrasound energy.

In any of the embodiments disclosed herein, the reactive agent(s) may be combined with non reactive carrier. For example the nonreactive carrier may be an acoustically transparent carrier such as ultrasound gel, castor oil, honey or the like.

In any of the embodiments disclosed herein the activation element, if provided, may include one or more of a light emitting diode and a heating element.

According to one embodiment, a composition is disclosed which includes at least one reactive agent in a nonreactive acoustically transparent carrier wherein the at least one reactive agent is responsive to energy exposure and provides indication of at least one of a duration of treatment, intensity of energy exposure, and peak temperature. Each reactive agent may include one or more of a thermochrome and a chromaphore. If the composition include two or more reactive agents, two or more may be thermochromes, two or more may be may be chromaphores or a combination of thermochromes and chromaphores may be provided. The reactive agent may provide indication of the peak temperature of the treatment area, the duration of treatment, or a combination thereof.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. A method for monitoring the amount of treatment delivered by a treatment device, comprising:
providing a treatment device including at least one activation element;
applying at least one reactive agent to a treatment area, and
actuating the treatment device and the activation element to deliver a treatment to a treatment area, wherein the at least one reactive agent reacts to energy delivered by the at least one activation element.

2. The method of claim 1, wherein the at least one reactive agent provides an indication of whether the treatment area has been treated.

3. The method of claim 1, wherein the at least one reactive agent provides an indication of a duration of treatment provided to the treatment area.

4. The method of claim 1, wherein a selected reactive agent provides an indication of a peak temperature and another reactive agent provides an indication of a duration of treatment in the treatment area.

5. The method of claim 1, wherein the at least one reactive agent provides an indication of a peak temperature of the treatment area.

6. The method of claim 1, wherein the treatment device is the activation element.

7. The method of claim 1, wherein each reactive agent includes at least one of a thermochrome and a chromaphore.

8. A method for monitoring the amount of treatment delivered by a treatment device, comprising:
providing a treatment device;
applying at least one reactive agent to a treatment area, and
actuating the treatment device to deliver treatment to a treatment area, wherein the at least one reactive agent reacts to energy delivered by the treatment device.

9. A system for monitoring treatment parameters, comprising:
a treatment device for delivering a treatment to a treatment area;
at least one activation element operably coupled to the treatment device such that the activation element is actuated each time the treatment device is actuated; and
at least one reactive agent applied to the treatment area and responsive to an energy transmitted from the at least one activation element.

10. The system of claim 9, wherein each of the at least one reactive agent includes at least one of a thermochrome and a chromaphore.

11. The system of claim 9, comprising two different reactive agents selected from the group of a thermochrome and a chromaphore.

12. The system of claim 9, wherein the at least one reactive agent provides an indication of whether a given treatment area has been treated.

13. The system of claim 9, wherein the at least one reactive agent provides an indication of the duration of treatment provided to a given treatment area.

14. The system of claim 9, wherein the at least one reactive agent provides an indication of a duration of treatment and peak temperature of the treatment area.

15. The system of claim 9, wherein the at least one reactive agent provides an indication of a peak temperature of the treatment area.

16. The system of claim 9, wherein the treatment device is an ultrasound transducer and the at least one reactive agent is nonreactive to ultrasound energy.

17. The system of claim 9, wherein the at least one further where the reactive agent is combined with an acoustically transparent carrier.

18. The system of claim 9, wherein the activation element is one of a light emitting diode and a heat source.

19. The system of claim 9, wherein the treatment device is the activation element.

20. A system for monitoring treatment parameters, comprising:
a treatment device for delivering a treatment to a treatment area; and
a reactive agent applied to the treatment area and responsive to an energy transmitted from the treatment device.

21. A composition, comprising:
at least one reactive agent in a nonreactive acoustically transparent carrier, wherein the at least one reactive agent is responsive to an energy exposure and provides an indication of at least one of a duration and intensity of the energy exposure.

22. The composition of claim 21, wherein the at least one reactive agent includes at least one of a thermochrome and a chromaphore.

23. The composition of claim 21, wherein the composition includes at least two reactive agents, each said reactive agent selected from the group of a thermochrome and a chromaphore.

24. The composition of claim 21, wherein the at least one reactive agent provides an indication of whether a given treatment area has been treated.

25. The composition of claim 21, wherein the at least one reactive agent provides an indication of a peak temperature of the treatment area.

26. The composition of claim 21, wherein the at least one reactive agent provides an indication of a duration of a treatment within a treatment zone.

27. A method for monitoring the amount of a treatment delivered by a treatment device, comprising:
providing a treatment device;
applying at least one reactive agent to a treatment area, each reactive agent reacting to an energy delivered by the treatment device; and
actuating the treatment device to deliver a treatment.
